(19) United States
(12) Patent Application Publication
(10) Pub. No.: US 2021/0207290 A1
(43) Pub. Date: Jul. 8, 2021

(54) THERMOSENSETIVE NANOFIBROUS STRUCTURE FOR EXOSOME ISOLATION

(71) Applicant: Matin Mahmoudifard, Karaj (IR)

(72) Inventor: Matin Mahmoudifard, Karaj (IR)

(73) Assignees: Matin Mahmoudifard, Karaj (IR); National institute of genetic engineering and biotechnology, Tehran (IR)

(21) Appl. No.: 17/206,567

(22) Filed: Mar. 19, 2021

Related U.S. Application Data
(60) Provisional application No. 63/070,287, filed on Aug. 26, 2020.

(51) Int. Cl. D01D 5/00; D01D 5/34; D04H 3/002; D04H 3/007

(52) U.S. Cl. CPC D01D 5/003; D01D 5/34; D10B 2321/10; D04H 3/007; D04H 3/002

(57) ABSTRACT

A method for isolating exosomes from a biological sample may include synthesizing a first nanofibrous substrate by coaxial electrospinning of a core polymer solution and a shell polymer solution. The shell polymer solution may include gelatin. A method for isolating exosomes may further include obtaining a second nanofibrous substrate by immobilizing an antibody, such as CD63, CD9, and CD81 to the first nanofibrous substrate, capturing the exosomes from the biological sample by incubating the biological sample with the second nanofibrous substrate, and releasing the captured exosomes from the second nanofibrous substrate by incubating the second nanofibrous substrate with water at a temperature of 37° C.

### CROSS-REFERENCE TO RELATED APPLICATION

This application claims the benefit of priority from pending U.S. Provisional Patent Application Ser. No. 63/070,287, filed on Aug. 26, 2020, and entitled “EXOSOME ISOLATION BY THERMOSENSITIVE NANOFIBER,” which is incorporated herein by reference in its entirety.

### TECHNICAL FIELD

The present disclosure generally relates to thermosensitive nanofibers, particularly to thermosensitive nanofibrous structures utilized for isolating exosome. More particularly, the present disclosure relates to coaxial thermosensitive nanofibrous structures utilized as substrates for exosome isolation and methods for producing such coaxial thermosensitive nanofibrous structures.

### BACKGROUND

Exosomes are small vesicular bodies released by various types of cells, including cancerous cells. They contain organic compounds of cells, like mRNAs and proteins. Furthermore, exosomes may mediate signal transduction in both autocrine and paracrine fashion by the transfer of proteins and RNA. from releasing cells to recipient cells. Exosomes may deliver signals from cancer cells to stroma cells or other cells from specific organs by being taken up by these stroma cells or other cells from specific organs, Consequently, such stroma cells or other cells from specific organs may form the pre-metastatic niche. Exosomes exist in human body fluids, such as in blood, urine, and saliva, which may make exosomes ideal markers for a noninvasive diagnostic method.

However, current methods for isolating exosomes are not efficient enough and this may limit the use of exosomes for diagnostic purposes. These isolation methods include ultracentrifugation, filtration, commercial kits, and microfluidics. Nevertheless, the aforementioned isolation methods may have some disadvantages. For example, ultracentrifugation requires a large number of samples and is time-consuming, filtration may harm the integrity of exosomes, and commercial kits utilized for isolating the exosomes may have residual precipitation matrices. Furthermore, the aforementioned methods do not have the ability to differentiate between exosomes and other shed membranes, lipid structures, or retrovirus particles that may exist in body fluids. Such difficulty in differentiation may be due to the fact that exosomes and other shed membranes, lipid structures, or retrovirus particles may be of similar size and density. Consequently, developing a method that may focus on separating and purifying exosomes in a more specific and cost-time efficient manner is crucial.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description and the drawings.

According to one or more exemplary embodiments, the present disclosure is directed to a method for isolating exosomes from a biological sample. An exemplary method may include synthesizing a first nanofibrous substrate by coaxial electrospinning of a core polymer solution and a shell polymer solution. An exemplary shell polymer solution may include gelatin. An exemplary method may further include obtaining a second nanofibrous substrate by immobilizing an antibody to the first nanofibrous substrate. An exemplary antibody may include at least one of CD63, CD9, and CD81. An exemplary method may further include capturing exosomes from a biological sample by incubating an exemplary biological sample with an exemplary second nanofibrous substrate and releasing the captured exosomes from an exemplary second nanofibrous substrate by incubating the second nanofibrous substrate with water at a temperature of 37° C.

According to one or more exemplary embodiments, the present disclosure is directed to a nanofibrous substrate for isolating exosomes from a biological sample. An exemplary nanofibrous substrate may include a plurality of core-shell nanofibers, where each core-shell nanofiber of the plurality of core-shell nanofibers may include a core made of a first polymer and a shell made of gelatin. An exemplary nanofibrous substrate may further include an antibody immobilized on the plurality of core-shell nanofibers, where the antibody may be at least one of CD63, CD9, and CD81.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for illustration and description only and are not intended as a definition of the limits of the present disclosure. Embodiments of the present disclosure will now be described by way of example in association with the accompanying drawings in which:

FIG. 1 illustrates a schematic representation of a method for synthesizing a nanofibrous substrate by immobilizing exosome-specific antibodies on a core-shell nanofiber and isolating exosomes utilizing the nanofibrous substrate, consistent with one or more exemplary embodiments of the present disclosure;

FIG. 2 illustrates a flowchart of a method for isolating exosomes from a biological sample, consistent with one or more exemplary embodiments of the present disclosure;

FIG. 3 illustrates a schematic view of a coaxial electrospinning apparatus, consistent with one or more exemplary embodiments of the present disclosure;

FIG. 4A illustrates a scanning electron microscope (SEM) image of the synthesized core-shell nanofibrous substrate, consistent with one or more exemplary embodiments of the present disclosure;

FIG. 4B illustrates a transmission electron microscope (TEM) image of the synthesized core-shell nanofibrous substrate, consistent with one or more exemplary embodiments of the present disclosure;

FIG. 5 illustrates ultraviolet-visible (UV-vis) spectra of a water sample in which core-shell nanofibrous substrate was incubated at 37° C., a water sample in which core-shell nanofibrous substrate was incubated at room temperature, and pure gelatin solution at room temperature, consistent with one or more exemplary embodiments of the present disclosure;

FIG. 6 illustrates an SEM image of the synthesized core-shell nanofibrous substrate after exosome isolation, consistent with one or more exemplary embodiments of the present disclosure;

FIG. 7A illustrates an SEM image of the synthesized core-shell nanofibrous substrate with exosome populations captured onto the synthesized core-shell nanofibrous substrate, consistent with one or more exemplary embodiments of the present disclosure; and

FIG. 7B illustrates an SEM image of core-shell nanofibrous membrane of FIG. 7A after being incubated with water at 37° C., consistent with one or more exemplary embodiments of the present disclosure.

### DETAILED DESCRIPTION

The novel features which are believed to be characteristic of the present disclosure, as to its structure, organization, use and method of operation, together with further objectives and advantages thereof, will be better understood from the following discussion.

The present disclosure is directed to exemplary embodiments of an exemplary nanofibrous substrate for isolating exosomes from a biological sample and exemplary embodiments of an exemplary method for synthesizing an exemplary nanofibrous substrate for isolating exosomes from a biological sample. An exemplary nanofibrous substrate may be made of core-shell nanofibers, on which an exemplary exosome-specific antibody, such as CD63, CD9, and CD81 may be immobilized. Such immobilization of exosome-specific antibodies onto an exemplary nanofibrous substrate may allow for obtaining a separation bed that may be highly specific for exosomes. Each exemplary nanofiber of exemplary core-shell nanofibers may include a shell of gelatin formed on a polymeric core. An exemplary antibody may be immobilized on an exemplary gelatin shell, either directly or by utilizing a cross-linking agent, such as 1-Ethyl-3-(3 -dimethylaminopropyl) carbodiimide/N-hydroxy succinimide (referred to hereinafter as “EDC/NHS”).

An exemplary gelatin shell of an exemplary core-shell nanofiber may dissolve in water at a biological temperature of approximately 37° C., which may allow for an easy release of exosomes captured by an exemplary nanofibrous substrate. In an exemplary embodiment, such dissolution of gelatin in water at an elevated temperature of 37° C. may allow for developing separation bed based on an exemplary second nanofibrous substrate with a gelatin shell that may be capable of holding exosomes during a washing process and capable of releasing captured exosomes after isolation without a need for extreme conditions. In other words, an exemplary second nanofibrous substrate with a gelatin shell may be capable of releasing captured exosomes specifically whenever an external force like heat is applied. Such external force may be provided by water at 37° C.

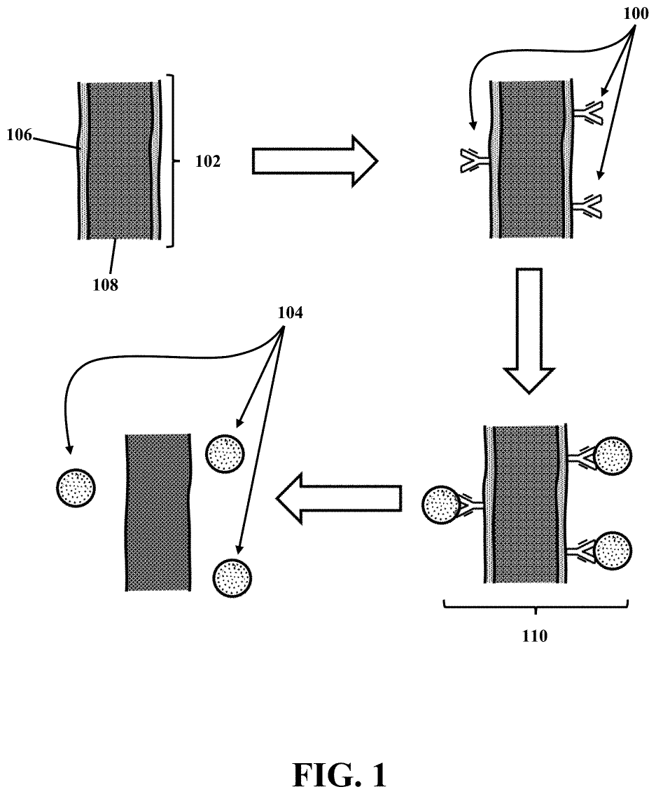

FIG. 1 illustrates a schematic representation of a method for synthesizing a nanofibrous substrate by immobilizing exosome-specific antibodies 100 on a core-shell nanofiber 102 and isolating exosomes 104 utilizing the nanofibrous substrate, consistent with one or more exemplary embodiments of the present disclosure. In an exemplary embodiment, an exemplary nanofibrous substrate, as described in the preceding paragraph may include core-shell nanofibers, such as core-shell nanofiber 102. An exemplary core-shell nanofiber, such as core-shell nanofiber 102 may include a shell 106 made of gelatin formed around a polymeric core 108. In an exemplary embodiment, exosome-specific antibodies, such as exosome-specific antibodies 100 may be immobilized on exemplary shells of exemplary core-shell nanofibers, such as shell 106 by incubating a solution of exosome-specific antibodies 100 and shell 106 In an exemplary embodiment, such immobilization of exosome-specific antibodies on surfaces of exemplary core-shell nanofibers may allow for capturing exosomes onto exemplary shells of exemplary core-shell nanofibers via exosome-specific antibodies. For example, exosomes may be captured onto shell 106 via exosome-specific antibodies 100, as illustrated in FIG. 1 (labeled with reference numeral 110). Gelatin may dissolve in water at a physiological temperature of approximately 37° C. Consequently, in order to release exemplary captured exosomes, exemplary core-shell nanofibers may be incubated with water at 37° C. In an exemplary embodiment, such incubation of exemplary core-shell nanofibers with water at 37° C. may lead to dissolution of exemplary gelatin shells of exemplary core-shell nanofibers, for example shell 106, and as a result exosomes 104 may be released into an exosome-rich solution (not illustrated). Since gelatin is utilized as a shell in a core-shell nanofibrous structure, dissolution of such thin layer of gelatin may create only a negligible amount of contamination in an exemplary exosome-rich solution.

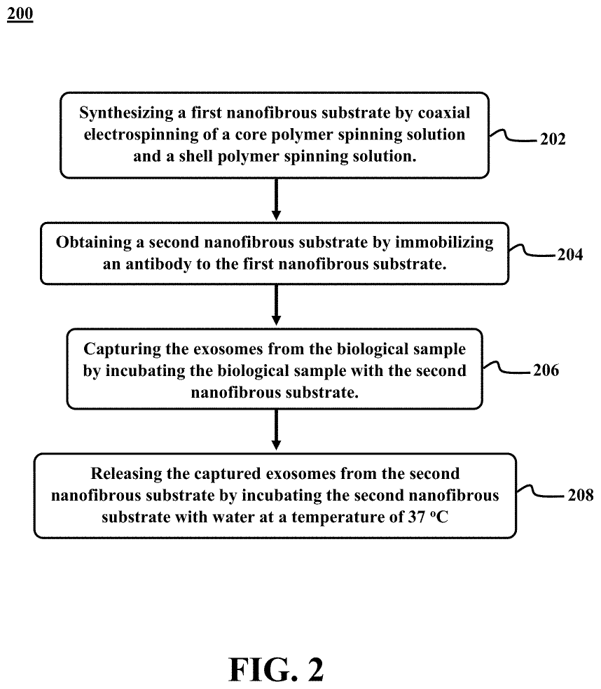

FIG. 2 illustrates a flowchart of a method 200 for isolating exosomes from a biological sample, consistent with one or more exemplary embodiments of the present disclosure. In an exemplary embodiment, method 200 may be similar to method 100. In an exemplary embodiment, method 200 may include a step 202 of synthesizing a first nanofibrous substrate by coaxial electrospinning of a core polymer solution and a shell polymer solution, a step 204 of obtaining a second nanofibrous substrate by immobilizing an antibody to the first nanofibrous substrate, a step 206 of capturing the exosomes from the biological sample by incubating the biological sample with the second nanofibrous substrate, and a step 208 of releasing the captured exosomes from the second nanofibrous substrate by incubating the second nanofibrous substrate with water at a temperature of 37° C.

In an exemplary embodiment, step 202 of synthesizing the first nanofibrous substrate may include coaxial electrospinning of a core polymer solution and a shell polymer solution. In an exemplary embodiment, core polymer solution may include a first polymer, such as at least one of poly acrylonitrile, poly ether sulfone, poly caprolactone, and poly lactic acid-co-glycolic acid. In an exemplary embodiment the shell polymer solution may include gelatin.

In an exemplary embodiment, the core polymer solution may include a solution of the first polymer in a suitable solvent. In an exemplary embodiment, the suitable solvent may include at least one of 1,1,1,3,3,3 hexafluoro-2-propanol (HIPF), 2,2,2-trifluoroethanol (TFE), and a mixture of water and acetic acid. In an exemplary embodiment, the core polymer solution may have a concentration between 8 w/v % and 13 w/v %. In an exemplary embodiment, the shell polymer solution may include a solution of gelatin in a suitable solvent. In an exemplary embodiment, the suitable solvent may include at least one of 1,1,1,3,3,3 hexafluoro-2-propanol (HIPF), 2,2,2-trifluoroethanol (TFE), and a mixture of water and acetic acid. In an exemplary embodiment, the shell polymer solution may have a concentration between 18 w/v % and 25 w/v %.

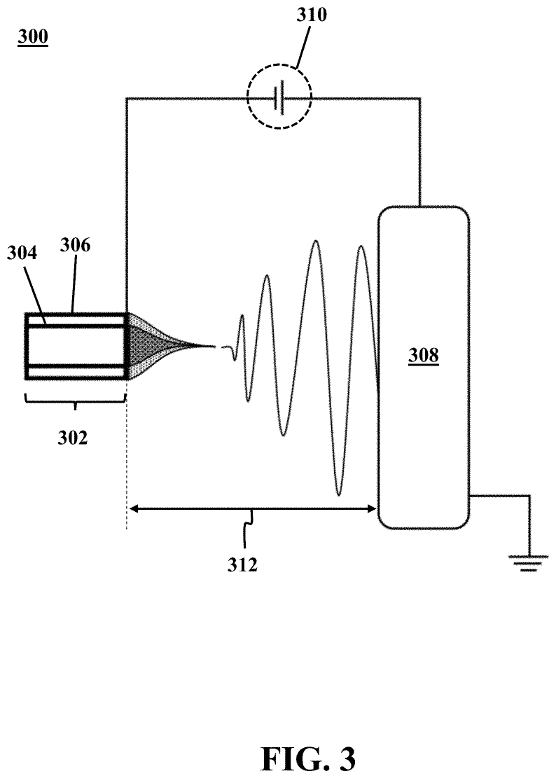

FIG. 3 illustrates a schematic view of a coaxial electrospinning apparatus 300, consistent with one or more exemplary embodiments of the present disclosure. In an exemplary embodiment, a coaxial electrospinning apparatus, such as coaxial electrospinning apparatus 300 may be utilized for performing step 202 of synthesizing the first nanofibrous substrate. In an exemplary embodiment, coaxial electrospinning apparatus 300 may include a coaxial electrospinning nozzle 302 that may include an inner nozzle 304, which may be coaxially disposed within an outer nozzle 306. In an exemplary embodiment, such coaxial electrospinning nozzle may allow for electrospinning of core-shell nanofibers, where a first spinning solution may be discharged from outer nozzle 306 to form a shell of a core-shell nanofiber, while a second spinning solution may be discharged from inner nozzle 304 to form a core of a core-shell nanofiber. For example, a shell polymer solution including gelatin may be discharged from outer nozzle 306 and a core polymer solution including a first polymer may be discharged from inner nozzle 304.

In an exemplary embodiment, coaxial electrospinning apparatus 300 may further include a collector 308 that may be positioned in front of coaxial electrospinning nozzle 302 at a predetermined distance 312 and a power supply 310 that may be connected between a tip of coaxial electrospinning nozzle 302 and collector 308. In an exemplary embodiment, the core polymer solution and the shell polymer solution may be fed into coaxial electrospinning nozzle 302 utilizing a pumping mechanism, such as a syringe pump. In an exemplary embodiment, coaxial arrangement of inner nozzle 304 and outer nozzle 306 of coaxial electrospinning nozzle 302 may allow for a coaxial injection of the core polymer solution and the shell polymer solution, where the core polymer solution may get drawn within the shell polymer solution to produce a continuous nanofiber of the core polymer coated with a layer of the shell polymer.

In an exemplary embodiment, the core polymer solution may be discharged from inner nozzle 304 and the shell polymer solution may be discharged from outer nozzle 306, where inner nozzle 304 and outer nozzle 306 may be positioned at predetermined distance 312 of between 15 cm and 25 cm from collector 308. In an exemplary embodiment, core-shell nanofibers produced this way may be collected on collector 308 as the first nanofibrous substrate.

In an exemplary embodiment, coaxial electrospinning of the core polymer solution and the shell polymer solution may further include applying a voltage between 13 kV and 18 kV between coaxial electrospinning nozzle 302 and collector 308. In an exemplary embodiment, inner nozzle 304 may have a nozzle gauge of between 18 and 21, while outer nozzle 306 may have a nozzle gauge of between 14 and 18. In an exemplary embodiment, the core polymer solution may be discharged out of inner nozzle 304 with a flowrate of 0.4 to 0.7 mLh⁻¹, while the shell polymer may be discharged out of outer nozzle 306 with a flowrate of 0.2 to 0.4 mLh⁻¹.

In an exemplary embodiment, step 204 of obtaining the second nanofibrous substrate may include immobilizing an exosome-specific antibody to the first nanofibrous substrate. In an exemplary embodiment, such immobilization of an exemplary exosome-specific antibody on the first nanofibrous substrate to obtain the second nanofibrous substrate may allow the second nanofibrous substrate to function as a bed for separating exosomes from biological samples exposed to the second nanofibrous substrate.

In an exemplary embodiment, immobilizing the exosome-specific antibody on the first nanofibrous substrate may include incubating a solution of an exosome-specific antibody, such as at least one of CD63, CD9, and CD81 with the first nanofibrous substrate. For example, a solution of an exosome-specific antibody with a concentration of between 5 μgmL⁻¹ and 20 μgmL⁻¹ in either water or phosphate-buffered saline (PBS) may be poured onto the first nanofibrous substrate. In an exemplary embodiment, incubation of the exosome-specific antibody and the first nanofibrous substrate may be carried out for a period of at most 24 hours at a temperature of approximately 4° C.

In an exemplary embodiment, step 204 of immobilizing the exosome-specific antibody on the first nanofibrous substrate may further include incubating a cross-linking agent with the first substrate, where the cross-linking agent may facilitate immobilization of the exosome-specific antibody on the first nanofibrous substrate in a later stage of step 204. In an exemplary embodiment, incubating the cross-linking agent with the first nanofibrous substrate may include pouring a solution of the cross-linking agent onto the first substrate. Such incubation of the solution of cross-linking agent and the first nanofibrous substrate may be carried out for a period of at least 15 minutes at a temperature of approximately 4° C. In an exemplary embodiment, the solution of the cross-linking agent may include an aqueous solution of EDC/NHS with a mass ratio of EDC to NHS between 1:1 and 1:2 (EDC:NHS). In an exemplary embodiment, the aqueous solution of EDC/NHS may have a mass ratio of EDC to NHS of either 1:1 or 1:1.5 (EDC:NHS). For example, for immobilizing an antibody, such as CD63 onto the first nanofibrous substrate, first an aqueous solution of EDC/NHS with a concentration between 0.5 mgL⁻¹ and 3 mgL⁻¹ may be incubated with the first nanofibrous substrate at 4° C. for at least 15 minutes. After that, excess EDC/NHS solution may be removed from the first nanofibrous substrate and then a solution of an exosome-specific antibody with a concentration of between 5 μgmL⁻¹ and 20 μgmL⁻¹ in either water or PBS may be poured onto the first nanofibrous substrate. As mentioned before, such utilization of a cross-linking agent may facilitate the immobilization of an exemplary antibody onto an exemplary first nanofibrous substrate to obtain a second nanofibrous substrate.

In an exemplary embodiment, the second nanofibrous substrate synthesized according to steps 202 and 204 may be utilized as a bed for isolating exosomes from a biological sample. As used herein, a biological sample may refer to a sample that may at least partly be obtained from a subject, such as a human.

As mentioned before, an exemplary shell of exemplary core-shell nanofibers of an exemplary first nanofibrous substrate may include gelatin, which may have hydroxyl and amine groups that may function as binding sites for an efficient immobilization of various molecules. In an exemplary embodiment, after immobilization of exosome-specific antibodies onto the first nanofibrous substrate to obtain the second nanofibrous substrate, some binding sites on the second nanofibrous substrate may still be vacant and ready for immobilization of molecules other that the exosome-specific antibodies. Consequently, in an exemplary embodiment, step 204 of immobilizing the exosome-specific antibody on the first nanofibrous substrate may further include blocking the remaining binding sites on the second nanofibrous substrate by incubating the second nanofibrous substrate with a bovine serum albumin (BSA) solution. In an exemplary embodiment, incubating the second nanofibrous substrate with the BSA solution may include pouring an aqueous solution of BSA with a concentration between 1 wt. % and 3 wt. % onto the second nanofibrous substrate. In an exemplary embodiment, incubation of the aqueous solution of BSA and the second nanofibrous substrate may be carried out at a temperature of approximately 4° C.

In an exemplary embodiment, step 206 of capturing the exosomes from the biological sample may include incubating the biological sample with the second nanofibrous substrate at a temperature of approximately 4° C. for a period of between 30 minutes and 1 hour. For example, the second nanofibrous substrate may be punched into small circular substrates that may be disposed within the wells of a multi-well plate. After that, a biological sample may be poured into the wells such that the biological sample may cover an entire surface of the punched circular substrates. After a period of 30 minutes to 1 hour, excess biological sample may be removed from the wells and then punched circular substrates may be washed with distilled water. Such incubation of a biological sample and the second nanofibrous substrates may lead to exosomes present in the biological sample to be captured onto the second nanofibrous substrate and thereby be separated from the biological sample.

In an exemplary embodiment, step 208 of releasing the captured exosomes from the second nanofibrous substrate may involve dissolving gelatin shell of the second nanofibrous substrate, onto which the exosomes are attached. In an exemplary embodiment, to dissolve the gelatin shell, the second nanofibrous substrate may be incubated with deionized water at a biological temperature of 37° C. In an exemplary embodiment, incubating the second nanofibrous substrate with water may include pouring water onto the second nanofibrous substrate and then incubating the water and the second nanofibrous substrate at a temperature of approximately 37° C. After a period of approximately 1 hour, gelatin shell may be dissolved in water and separated exosomes may be released to form an exosome-rich aqueous phase.

In an exemplary embodiment, such sensitivity of gelatin shell of exemplary core-shell nanofibers of an exemplary second nanofibrous substrate may allow for washing or rinsing an exemplary second nanofibrous substrate at temperatures lower than 37° C., while captured exosomes on an exemplary second nanofibrous substrate may easily be released by dissolving an exemplary gelatin shell at a biological temperature of 37° C. Such temperature-sensitivity of gelatin shell is the reason why core-shell nanofibers of exemplary embodiments may be referred to as thermosensitive nanofibers.

### EXAMPLE

In this example, a core-shell nanofibrous substrate is synthesized that may be utilized as a separation bed for isolating exosomes from a biological sample. The core-shell nanofibrous substrate was synthesized by a method similar to steps 202 and 204 of method 200. In this example, a core-shell nanofibrous substrate was synthesized with a core made of polycaprolactone (PCL) and a shell made of gelatin type A. Furthermore, mouse anti-human CD63 was immobilized on the core-shell nanofibrous substrate. Here, a fresh aqueous solution of EDC/NHS was incubated with the core-shell nanofibrous substrate to activate carboxyl group of gelatin shell for better binding of CD63 antibody molecules to the surface of gelatin shell.

In this example, the core-shell nanofibrous substrate was synthesized by utilizing a coaxial electrospinning apparatus similar to electrospinning apparatus 300. A core solution of 10 w/v % PCL in 2,2,2-Trifluoroethanol (TFE) and a shell solution of 20 w/v % gelatin in TFE were poured in two separate 5 ml syringe pumps. A coaxial electrospinning nozzle similar to coaxial electrospinning nozzle 302 with an inner nozzle similar to inner nozzle 304 and an outer nozzle similar to outer nozzle 306 may be utilized for coaxial electrospinning of the PCL solution and the gelatin solution. Here, the syringe pumps were utilized to pump the PCL solution out of inner nozzle 304 with a flowrate of 0.6 mL/h and the gelatin solution out of outer nozzle with a flowrate of 0.3 mL/h. In this example, inner nozzle 304 had a nozzle gauge of approximately 19 and outer nozzle 306 had a nozzle gauge of approximately 16. A power supply, similar to power supply 310 was utilized to apply a voltage of approximately 16 kV between a tip of coaxial electrospinning nozzle 302 and a collector similar to collector 308. Here, collector 308 may be positioned at a distance of 15 cm from coaxial electrospinning nozzle 302.

In this example, a fluorescent-type immunoassay was designed using the core-shell nanofibrous substrate to visualize the capability of the core-shell nanofibrous substrate to be used as a substrate for the capture and release of biomolecules. To this end, core-shell nanofibrous substrate was incubated with a mouse anti-human CD63 with a concentration of 5 μg/ml for 1 h. Then, the core-shell nanofibrous membrane was washed thoroughly utilizing PBS, twice. After that, the core-shell nanofibrous substrate was blocked by incubating the core-shell nanofibrous substrate with a BSA solution with a concentration of 1 wt. % for 1 h. Finally, the core-shell nanofibrous substrate was incubated for 1 h with an FITC-labeled anti-mouse IgG antibody solution at room temperature. The core-shell nanofibrous substrate was then washed five times with PBS to remove excess FITC-labeled antibody. For purpose of comparison, two similar samples of core-shell nanofibrous membranes were prepared. After conjugation with FITC-labeled antibody, both samples were immersed into 500 μl of deionized water, then a first sample of the two samples was incubated at room temperature and a second sample of the two samples was transferred to a microplte at 37° C. for 1 h. Finally, both samples were analyzed for fluorescence activity by fluorescent microscopy.

The morphologies of the as-synthesized core-shell nanofibrous substrate samples with and without exosome was investigated by scanning electron microscopy (SEM), after gold spattering on the core-shell nanofibrous substrate samples. Fourier transform infra-red (FT-IR) spectroscopy was utilized for investigating the chemical characteristics of the core-shell nanofibrous substrate samples. Furthermore, transmission electron microscopy (TEM) was utilized for investigating the morphology of the core-shell nanofibrous substrate samples and captured exosomes on the core-shell nanofibrous substrate samples. Finally, ultraviolet (UV)-visible absorbance study was performed on the water that contained the core-shell nanofibrous substrate samples.

In this example, a biological sample may be modeled by culturing PC3 cells in an RPMI medium supplemented with 10% FBS, 100 units/L penicillin, and 100 μg/mL streptomycin. The cultured cells were then incubated in a humidified 5% CO₂-containing balanced air incubator at 37° C. The medium was changed every two days. When the confluency of the cultured cells reached 80 to 90%, the PC3 cells were washed three times with PBS. Then, the culture medium was replaced with an FBS-free RPMI medium and was harvested for 48 hours to collect exosomes.

In this example, two approaches were taken for isolation of exosomes in the as-prepared biological sample. In a first approach, core-shell nanofibrous substrate of PCL/gelatin synthesized in this example was utilized as a separation bed for isolating the exosomes. In another approach, for sake of comparison, the exosomes were isolated utilizing an ultracentrifugation method.

The core-shell nanofibrous substrate was punched in the form of small circles to fit the wells of a 96-well microplate. The punched core-shell nanofibrous substrates were disposed within the wells and were washed twice with deionized water to remove any possible contaminants. A fresh aqueous solution of EDC/NHS containing 0.8 mg of EDC and 1.2 mg of NHS per ml was incubated with the core-shell nanofibrous substrate for 15 minutes to activate the carboxyl group of gelatin shell for better binding of antibody molecules to the surface of the core-shell nanofibrous substrate. Then 100 μl of an anti-CD63 antibody solution with a concentration of 5 μg/ml in PBS at a pH=7.4 was poured into each well and was incubated with the core-shell nanofibrous substrate overnight in a steady condition at 4° C., so that the antibodies may be bound to the surface of the core-shell nanofibrous substrate.

After removing excess antibody from the wells, the core-shell nanofibrous substrate was washed twice with PBS. In order to inhibit nonspecific adsorption of undesired molecules, 100 microliters of a 1 wt. % BSA solution at a pH=7.4 was poured into each well and the plates were incubated for one hour at 4° C. After removing BSA from the wells, the core-shell nanofibrous substrate was washed twice. The collected media from PC3 cells as the biological sample was poured on the antibody-conjugated core-shell nanofibrous substrate. To this end, 100 microliters of the biological sample was poured into each well of a 90-well microplate containing punched core-shell nanofibrous substrates.

Antibody-conjugated core-shell nanofibrous substrates without isolated exosomes may be assigned as a control group. After 1 hour, the media were removed from the wells and core-shell nanofibrous substrates were washed twice. Then, 100 microliter of deionized water was poured into each well and was incubated with the core-shell nanofibrous substrate punches at 37° C. for 1 h in order to release adsorbed exosomes on the surface of the core-shell nanofibrous substrates to the water. After one hour, the solution on the core-shell nanofibrous substrate may be transferred into other empty wells, in order to perform a direct ELISA test to assess the efficacy of exosome isolation by the antibody-conjugated core-shell nanofibrous substrates.

As mentioned before, for comparison, a biological sample made of obtained media from PC3 cells was first centrifuged for 10 minutes in 500×g to pellet the cell debris. After that, the supernatant was centrifuged again for 20 minutes in 20,000×g to remove larger vesicles and proteins. After that, filtration was carried out utilizing a 0.22-micron filter. Ultracentrifugation was performed for the final supernatant at 120,000×g for 2 hours to pellet the exosomes. The pellet was washed in a large volume of PBS to eliminate contaminating proteins and was centrifuged one last time at the same high speed of 120,000×g.

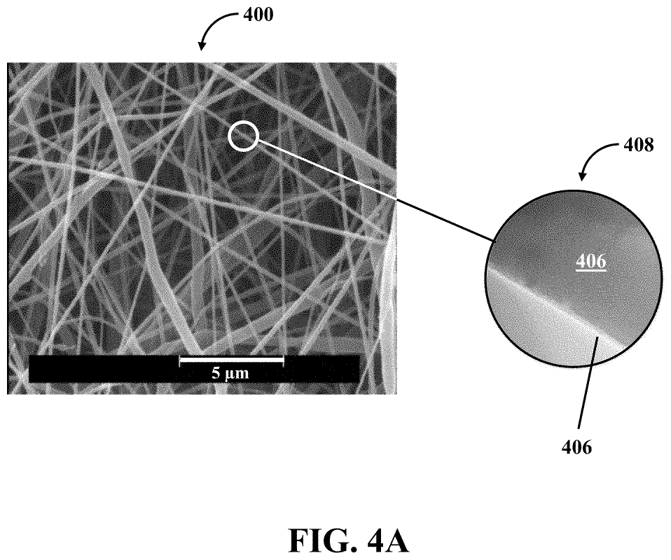

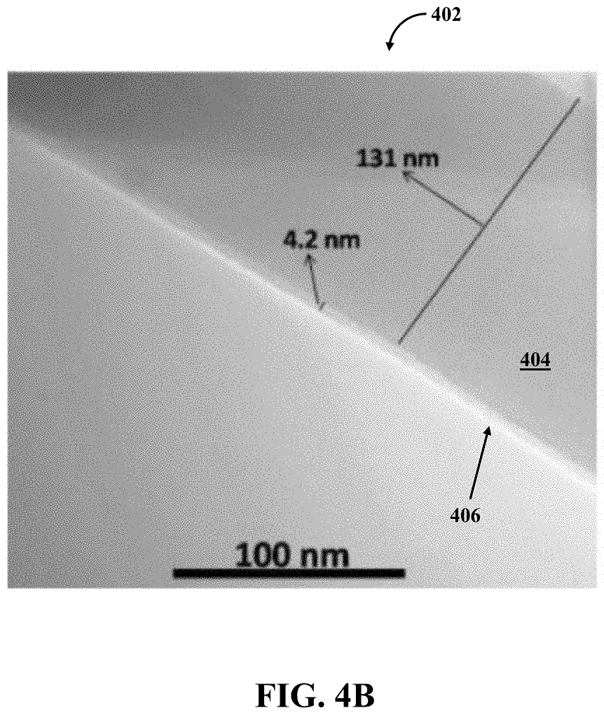

FIG. 4A illustrates a scanning electron microscope (SEM) image 400 of the synthesized core-shell nanofibrous substrate, consistent with one or more exemplary embodiments of the present disclosure. FIG. 4B illustrates a transmission electron microscope (TEM) image 402 of the synthesized core-shell nanofibrous substrate, consistent with one or more exemplary embodiments of the present disclosure. As evident in FIGS. 4A and 4B, the synthesized core-shell nanofibrous substrate have a highly uniform structure and an average diameter of the nanofibers are in the range of 290 nm. Referring to FIG. 4A, an inset 408 illustrates the detail of the core-shell structure of the synthesized core-shell nanofibrous substrate. As evident in inset 408, the nanofibers include a PCL core 404 coaxially coated by a gelatin shell 406. Furthermore, as evident in FIG. 4B, a distinct boundary between PCL core 404 and thin gelatin shell 406. Here, the thickness of gelatin shell 406 is approximately 10 nm.

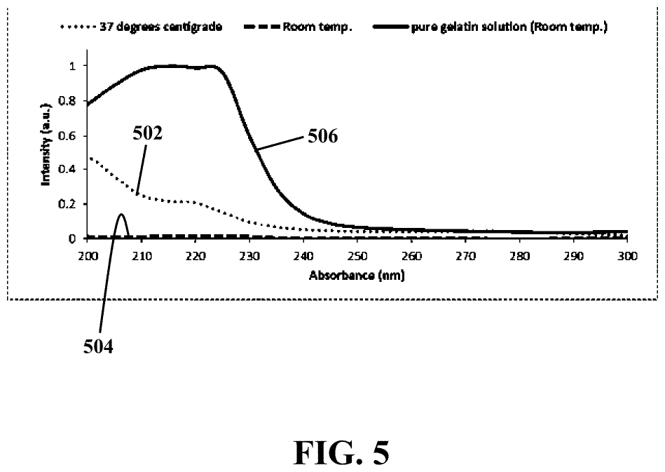

illustrates ultraviolet-visible (UV-vis) spectra of a water sample in which core-shell nanofibrous substrate was incubated at 37° C. (502), a water sample in which core-shell nanofibrous substrate was incubated at room temperature (504), and pure gelatin solution at room temperature (506), consistent with one or more exemplary embodiments of the present disclosure. UV-vis spectra for the water samples were obtained after incubating the core-shell nanofibrous substrate in the water samples at two different temperatures of 37° C. and room temperature for 1 hour. The absorption curve for water is measured and reported. This was done to investigate the gelatin shell releasability at biological temperature of 37° C. As evident in FIG. 5, pure gelatin solution (curve 506) shows an absorption peak at 220 nm. The same absorbance peak may also be observed for the solution obtained from incubation of core-shell nanofibrous substrate with water at 37° C. (curve 502). Such similar absorbance peak may indicate that at 37° C., the gelatin shell of core-shell nanofibrous substrate was dissolved in water, while at a temperature lower than 37° C., for example, the tested room temperature, the amount of gelatin released into water was observed to be insignificant.

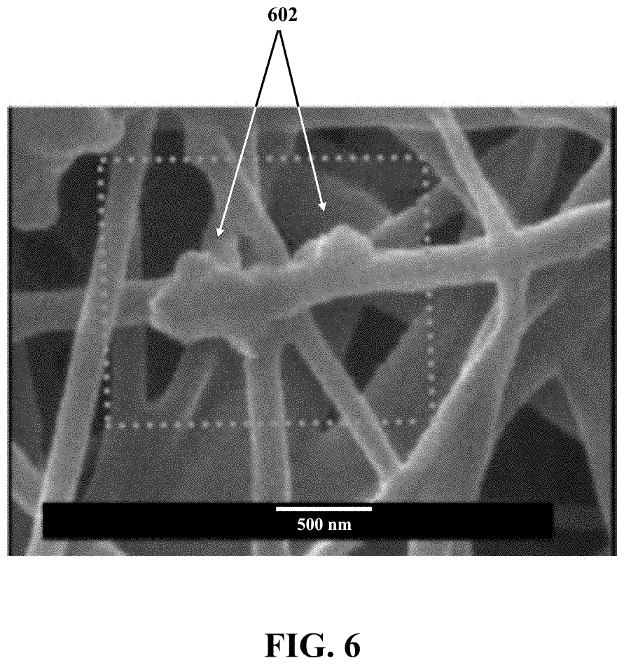

FIG. 6 illustrates an SEM image of the synthesized core-shell nanofibrous substrate after exosome isolation, consistent with one or more exemplary embodiments of the present disclosure. Exosomes populations 602 that were captured on the surface of the core-shell nanofibrous substrate are visible. This shows a successful separation of exosomes from the biological sample utilizing the core-shell nanofibrous substrate.

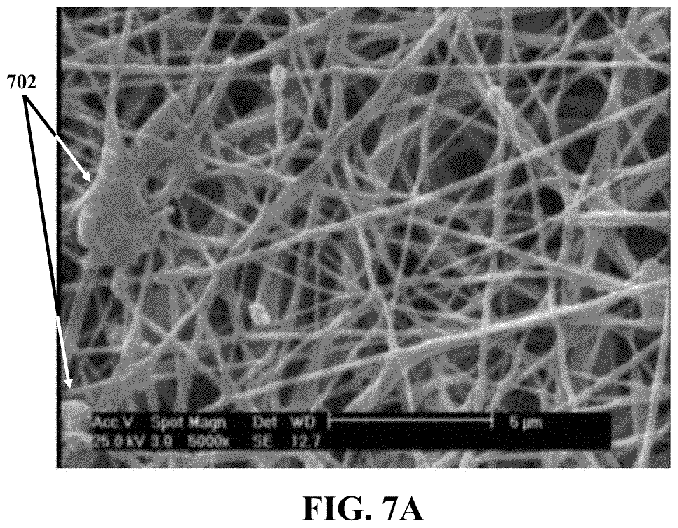

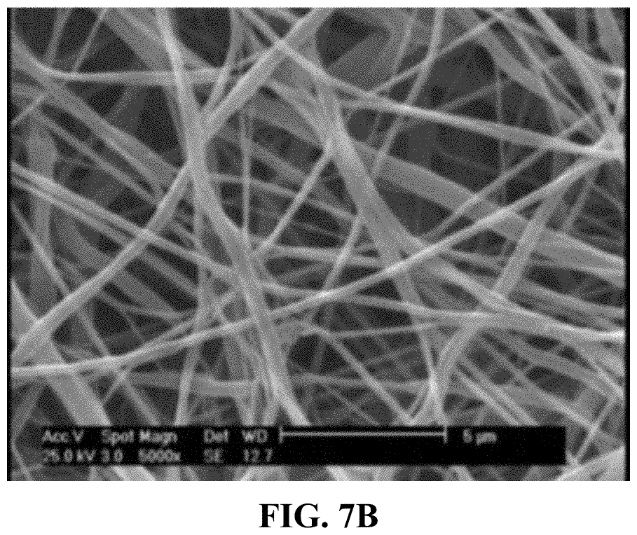

FIG. 7A illustrates an SEM image of the synthesized core-shell nanofibrous substrate with exosome populations 702 captured onto the synthesized core-shell nanofibrous substrate, consistent with one or more exemplary embodiments of the present disclosure. FIG. 7B illustrates an SEM image of the same core-shell nanofibrous membrane after being incubated with water at 37° C., consistent with one or more exemplary embodiments of the present disclosure. As evident, after incubation at 37° C. approximately all the captured exosomes are released from the surface along with the dissolution of the gelatin shell.

As mentioned before, indirect ELISA technique was utilized for investigating the exosome isolation efficacy of the synthesized core-shell nanofibrous substrate of the present example. ELISA signal confirmed that approximately more than 87% of exosomes which existed in the cell media were successfully captured and released by the synthesized core-shell nanofibrous substrate of the present example. As mentioned before, exosome isolation was further performed by an ultracentrifugation method for comparison. The exosome isolation efficacy for the ultracentrifugation method was approximately 98%, while the efficacy of the isolation method of this example was approximately 87%, which is comparable to that of the ultracentrifugation.

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps.

Moreover, the word “substantially” when used with an adjective or adverb is intended to enhance the scope of the particular characteristic, e.g., substantially planar is intended to mean planar, nearly planar and/or exhibiting characteristics associated with a planar element. Further use of relative terms such as “vertical”, “horizontal”, “up”, “down”, and “side-to-side” are used in a relative sense to the normal orientation of the apparatus.

What is claimed is:

1. A method for isolating exosomes from a biological sample, the method comprising:
synthesizing a first nanofibrous substrate by coaxial electrospinning of a core polymer spinning solution and a shell polymer spinning solution, the shell polymer spinning solution comprising gelatin;
obtaining a second nanofibrous substrate by immobilizing an antibody to the first nanofibrous substrate, the antibody comprising at least one of CD63, CD9, and CD81;
capturing the exosomes from the biological sample by incubating the biological sample with the second nanofibrous substrate; and
releasing the captured exosomes from the second nanofibrous substrate by incubating the second nanofibrous substrate with water at a temperature of 37° C.

2. The method of claim 1, wherein the coaxial electrospinning of the core polymer spinning solution and the shell polymer spinning solution comprises coaxial electrospinning of the core polymer spinning solution and gelatin dissolved in a solvent with a concentration between 18 w/v % and 25 w/v %.

3. The method of claim 2, wherein the core polymer spinning solution comprises a first polymer dissolved in the solvent with a concentration between 8 w/v % and 13 w/v %.

4. The method of claim 3, wherein the first polymer comprises at least one of poly acrylonitrile, poly ether sulfone, poly caprolactone, and poly lactic acid-co-glycolic acid.

5. The method of claim 4, wherein the shell polymer spinning solution comprises gelatin dissolved in the solvent, the solvent comprising at least one of 1,1,1,3,3,3 hexafluoro-2-propanol (HIPF), 2,2,2-trifluoroethanol (TFE), and a mixture of water and acetic acid.

6. The method of claim 3, the coaxial electrospinning comprises discharging the core polymer spinning solution from a first electrospinning nozzle of a coaxial nozzle positioned at a distance between 15 cm and 25 cm from a collector, discharging the shell polymer spinning solution from a second electrospinning nozzle of the coaxial nozzle, the second nozzle coaxially disposed within the first nozzle, applying a voltage between 13 kV and 18 kV between the coaxial nozzle and the collector.

7. The method of claim 6, wherein coaxial electrospinning of the core polymer spinning solution and the shell polymer spinning solution comprises discharging the core polymer spinning solution from the first electrospinning nozzle of the coaxial nozzle and discharging the shell polymer spinning solution from the second electrospinning nozzle of the coaxial nozzle, wherein the first electrospinning nozzle has a nozzle gauge between 18 and 21 and the second electrospinning nozzle has a nozzle gauge between 14 and 18.

8. The method of claim 3, wherein immobilizing the antibody to the first nanofibrous substrate comprises incubating a solution of the antibody with the first nanofibrous substrate, wherein the solution of the antibody has a concentration of 5 μg/mL to 20 μg/mL.

9. The method of claim 8, wherein immobilizing the antibody to the first nanofibrous substrate comprises incubating the solution of the antibody with the first nanofibrous substrate at a temperature of 4° C. for at most 24 hours.

10. The method of claim 8, wherein immobilizing the antibody to the first nanofibrous substrate further comprises incubating a solution of a cross-linking agent with the first nanofibrous substrate, the solution of the cross-linking agent comprising a 1-Ethyl-3-(3-dimethylaminopropyl) carbodiimide (EDC)/N-hydroxy succinimide (NHS) aqueous solution with a concentration between 0.5 mgL⁻¹ and 3 mgL⁻¹.

11. The method of claim 10, wherein immobilizing the antibody to the first nanofibrous substrate further comprises incubating the solution of the cross-linking agent with the first nanofibrous substrate, the solution of the cross-linking agent comprising the EDC/NHS aqueous solution with a mass ratio of EDC to NHS between 1:1 and 1:2 (EDC:NHS).

12. The method of claim 8, further comprising blocking the second nanofibrous substrate by incubating the second nanofibrous substrate with a bovine serum albumin (BSA) solution, wherein the BSA solution has a concentration between 1 and 3 wt. %.

13. The method of claim 12, wherein blocking the second nanofibrous substrate comprises incubating the second nanofibrous substrate with the BSA solution at 4° C.

14. The method of claim 12, wherein capturing the exosomes from the biological sample by incubating the biological sample with the second nanofibrous substrate at 4° C. for 30 minutes to 1 hour.

15. The method of claim 14, wherein the core polymer spinning solution comprises the first polymer dissolved in the solvent, the first polymer comprising at least one of poly acrylonitrile, poly ether sulfone, poly caprolactone, and poly lactic acid-co-glycolic acid.

16. The method of claim 15, wherein the shell polymer spinning solution comprises gelatin dissolved in the solvent, the solvent comprising at least one of 1,1,1,3,3,3 hexafluoro-2-propanol (HIPF), 2,2,2-trifluoroethanol (TFE), and a mixture of water and acetic acid.

17. The method of claim 16, wherein the coaxial electrospinning comprises discharging the core polymer spinning solution from a first electrospinning nozzle of a coaxial nozzle positioned at a distance between 15 cm and 25 cm from a collector, discharging the shell polymer spinning solution from a second nozzle of the coaxial nozzle, the second nozzle coaxially disposed within the first nozzle, applying a voltage between 13 kV and 18 kV between the coaxial nozzle and the collector.

18. A nanofibrous substrate for isolating exosomes from a biological sample, the nanofibrous substrate comprising:
a plurality of core-shell nanofibers, each core-shell nanofiber of the plurality of core-shell nanofibers comprising a core made of a first polymer and a shell made of gelatin; and
an antibody immobilized on the plurality of core-shell nanofibers, the antibody comprising CD63, CD9, and CD81.

19. The nanofibrous substrate of claim 18, wherein the antibody is immobilized on the plurality of core-shell nanofibers utilizing a cross-linking agent, the cross-linking agent comprising an EDC/NHS cross-linking agent with a mass ratio of EDC to NHS between 1:1 and 1:2 (EDC:NHS).

20. The nanofibrous substrate of claim 19, wherein the plurality of core-shell nanofibers are further blocked by BSA.
